(19) United States
(12) Patent Application Publication
(10) Pub. No.: US 2009/0226516 A1
(43) Pub. Date: Sep. 10, 2009

(54) SARTAN COMPOSITIONS

(75) Inventors: Benoit Schmitt, Irvine, CA (US); Laurent Imler, Irvine, CA (US)

Correspondence Address: HARNESS, DICKEY & PIERCE, P.L.C., P.O. BOX 828, BLOOMFIELD HILLS, MI, 48303, US

(73) Assignee: Pharma PASS II LLC, Irvine, CA (US)

(21) Appl. No.: 12/042,003

(22) Filed: Mar. 4, 2008

(51) Int. Cl. A61K 9/20; A61K 31/41; A61P 9/12; A61P 9/00; A61P 9/04

(52) U.S. Cl. 424/465; 514/381

(57) ABSTRACT

The invention provides a sartan or salt thereof composition having a dissolution in acetate buffer at pH 4.0 using USP type I method basket 10 Mesh at 75 rpm, of at least 20% after 90 minutes, 50% after 160 minutes, and 70% after 240 minutes

### FIELD OF THE INVENTION

The invention relates to an improved sartan compositions, and to a preparation process thereof. Especially the invention is a valsartan composition.

### BACKGROUND OF THE INVENTION

Sartans are currently among the most therapeutically effective drugs available for modulating the rennin-angiotension-aldosterone system in the treatment of hypertension, diabetic nephropathy and congestive heart failure. Specific examples of sartans include, inter alia, valsartan, candesartan, eprosartan, telmisartan, losartan, irbesartan, and olmesartan. The mechanism of action of sartans has been elucidated in some detail. It is believed that sartans block the activation of angiotensin II AT₁ receptors, causes directly vasodilatation, reduces secretion of vasopressin as well as secretion and production of aldosterone, the combination of which leads to the reduction of blood pressure. The AT₁ receptor is the best elucidated angiotensin receptor. Effects mediated by the AT₁ receptor include vasoconstriction, aldosterone synthesis and secretion, but also the vasopressin secretion, cardiac hypertrophy, augmentation of peripheral noradrenergic activity, vascular smooth muscle cells proliferation, decreased renal blood flow, renal renin inhibition, renal tubular sodium reuptake, modulation of central sympathetic nervous system activity, cardiac contractility, central osmocontrol and extracellular matrix formation.

Valsartan, one of the sartans is the common name of the chemical compound 3-methyl-2-[pentanoyl-[[4-[2-(2H-tetrazol-5-yl)phenyl]phenyl]methyl]amino]-butanoic acid, disclosed in U.S. Pat. No. 5,399,578 owned by Ciba Geigy Corp. and sold under various trademarks such as DIOVAN®, TAREG® (Novartis) or NISIS (Beaufour Ipsen Pharma)

Eudragit® polymers are well-known in the pharmaceutical industry, and are used typically as coatings for tablets. US2005/0277691 discloses modified release composition comprising an outer coating applied as a membrane. Eudragit® polymers are mentioned as possible coating materials.

Patent applications WO2007/052307 and WO2005/079748 disclose stabilized valsartan, losartan and candesartan compositions comprising an outer coating based on methacrylic acid such as Eudragit. US2003/035832 and WO2005/041941 disclose a solid valsartan oral form, which contains hydrochlorothiazide to provide improved stability properties and allows direct tabletting.

In patent application WO 2006/113631, the aim of the invention is to provide an increased bioavailable and soluble valsartan composition at low pH, which comprises apart from the active ingredient, a solubility enhancing agent. However, disclosed enhancing agents are surfactants, solubilizers, complexing agents, or hydrotropic agents. Methacrylic acid polymers, and esters thereof are only referred as polymeric release retardant materials.

Patent application WO 2006/044202, also discloses compositions providing enhanced release of the drug within the upper intestinal tracks, comprising a core with losartan as the active ingredient, coated with at least one enteric material such as Eudragit copolymers.

However, these sartans compositions of the prior art still have limited solubility in acidic medium such as in the gastric juice, where they usually show an incomplete dissolution. The bioavailability of the drug is thus reduced.

### SUMMARY OF THE INVENTION

The invention allows achieving high solubility in the acidic medium of the stomach, which in turn allows plasmatic concentration sufficient to achieve the therapeutical effects.

The invention provides a sartan or salt thereof composition having a dissolution in acetate buffer at pH 4.0 using USP type I method basket 10 Mesh at 75 rpm, of at least 20% after 90 minutes, 50% after 160 minutes, and 70% after 240 minutes.

According to another embodiment, the sartan or salt thereof composition has a dissolution in acetate buffer at pH 4.0 using USP type I method basket 10 Mesh at 75 rpm, of at least 60% after 90 minutes, 80% after 160 minutes and 90% after 240 minutes.

According to another embodiment, the sartan or salt thereof comprises between 10 and 40% wt of sartan.

According to another embodiment, the sartan is selected from the group consisting of valsartan, candesartan, eprosartan, telmisartan, losartan, irbesartan, and olmesartan, and salts thereof.

According to another embodiment, the composition is in a tablet form.

Preferably, the sartan or salt thereof is valsartan.

The invention also provides a composition comprising at least one aminoalkyl methacrylate copolymer dispersed therein.

Preferably, the aminoalkyl methacrylate copolymer is dimethyl aminoethyl methacrylate.

According to another embodiment, the composition comprises between 10 and 80% wt of aminoalkyl methacrylate copolymer.

According to another embodiment, the sartan is selected from the group consisting of valsartan, candesartan, eprosartan, telmisartan, losartan, irbesartan, and olmesartan, and salts thereof, and preferably is valsartan.

According to another embodiment, the composition is in a tablet form.

The invention further provides a method for the treatment of hypertension, diabetic nephropathy or congestive heart failure, comprising the step of orally administering to a patient in need of such treatment a therapeutically effective unit dosage of sartan or salt thereof composition having a dissolution in acetate buffer at pH 4.0 using USP type I method basket 10 Mesh at 75 rpm, of at least 50% after 160 minutes.

The invention also provides a method for the treatment of hypertension, diabetic nephropathy or congestive heart failure, comprising the step of orally administering to a patient in need of such treatment a therapeutically effective unit dosage of a sartan or salt thereof composition comprising at least one aminoalkyl methacrylate copolymer dispersed therein.

The invention further provides a method for the manufacture of a sartan composition, comprising the steps of (i) preparing granules comprising a sartan and at least one aminoalkyl methacrylate copolymer mixed therewith, and (ii) compressing the resulting granules into a tablet.

### BRIEF DESCRIPTION OF THE DRAWINGS

FIG. 1 is a representation of the solubility of compositions of the invention and a composition of the prior art in an acidic medium.

FIG. 2 is a representation of a process for the preparation of a spray-dried active powder.

FIG. 3 is a representation of a process for the preparation of the tablet core.

FIG. 4 is a representation of a process for the preparation of coatings.

### DETAILED DESCRIPTION OF EMBODIMENTS OF THE INVENTION

The term “sartan” as used in the invention designates at least one angiotensin II AT₁ receptors antagonist. The sartan is preferably selected from the group consisting of, valsartan, candesartan, eprosartan, telmisartan, losartan, irbesartan, and olmesartan, and salts thereof. Preferred sartans are the commercial sartans, such as losartan, irbesartan and candesartan, and especially valsartan.

Sartans can be used in the pharmaceutical compositions of the present invention either as the free acid or as any pharmaceutically acceptable salt thereof. The free acid can be prepared, for example, by hydrolysis of the corresponding lactone form or by treatment of the salt form of the acid with cationic exchange resin and evaporating the water portion. The free acid can be used to form the pharmaceutically acceptable salt form, by conventional methods known in the art. Among preferred pharmaceutically acceptable salts are metal and amine salts. The term “pharmaceutically acceptable metal salt” thus includes, but is not limited to, sodium, potassium, lithium, calcium, magnesium, aluminum, iron, or zinc salts. Such salts may be derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide, salts formed by reaction with ammonium hydroxide or organic amine salt or for example methylglucamine, choline, arginine, 1-deoxy-2-(methylamino)-D-glucitol and the like. A preferred salt is a metal salt such as the sodium or calcium salt.

The amount of the active ingredient in the pharmaceutical compositions of the present invention will be a therapeutically effective amount. A therapeutically effective amount will generally be an amount within the range of from about 0.05 to about 70%, and preferably an amount within the range of from about 1 to about 60% by weight of the composition. It is understood that higher or lower weight percentages of the active ingredient may be present in the pharmaceutical compositions. By “therapeutically effective amount” as used herein is meant an amount of active component in the pharmaceutical compositions of the present invention which is effective to beneficially treat the patient in need thereof. An especially adapted range for the active ingredient content is 10 to 40% by weight.

The second component of the composition of the invention is the aminoalkyl methacrylate copolymer which is dispersed therein. Such polymer is a known polymer and is available as the Eudragit®E100 from Rohm & Haas. The inventor has found that surprisingly, when the polymer is used dispersed in the composition instead of used as a coating, a composition with enhanced solubility in acidic medium is obtained.

Said polymer is preferably a dimethyl aminoethyl methacrylate, and preferably is EudragitE100.

The pharmaceutical compositions of the present invention will generally contain Eudragit polymer between about 10 to about 80% by weight percent; preferably between about 20 to about 70 wt %; and even more preferably between 40 and 60 wt % by weight of the pharmaceutical composition. A particularly preferred percentage of the Eudragit polymer is about 50%.

The invention thus provides a pharmaceutical composition having an enhanced solubility in acidic medium. Preferably, the composition exhibits a dissolution in acetate buffer at pH 4.0, using the US Pharmacopeia type I method, basket 10 Mesh at 75 rpm, of at least 20% after 90 minutes, 50% after 160 minutes, and 70% after 240 minutes, preferably of at least 40% after 90 minutes, 70% after 160 minutes, and 80% after 240 minutes, and more preferably of at least 60% after 90 minutes, 80% after 160 minutes, and 90% after 240 minutes.

Such values could not be reached with the formulations of the prior art.

The preferred dosage forms of the pharmaceutical compositions of the present invention are solid dosage forms adapted for oral administration. Tablet dosage forms are the particularly preferred solid dosage forms of the stabilized pharmaceutical compositions of the present invention. Tablet dosage forms may contain for example, as excipients, any pharmaceutically acceptable lubricant, binder, disintegrant, diluent, carrier, preservative or combination thereof. Solid dosage forms that are not formulated as tablets typically do not need a lubricant component since this is typically added to facilitate manufacture of tablet dosage forms. For the purpose of oral preparations of the present invention, pharmaceutically acceptable inert carriers or diluent or filler can be either solid or not. Among other preferred dosage forms useful for formulating the stabilized pharmaceutical compositions of the present invention include powders, dispersible granules, capsules and cachets.

The pharmaceutical compositions of the present invention may also contain any pharmaceutically acceptable excipient or combination thereof. Conventional pharmaceutical excipients include those which function in a dosage form, for example, as a lubricant, glidant, diluent, binder, disintegrant, carrier, colorant, preservative or coating material.

Examples of pharmaceutically acceptable excipients include, but are not limited to, lactose, dextrates, dextrin, dextrose, mannitol, dicalcium phosphate, xylitol, sugar, saccharose, corn starch, hydrolyzed starch (malto-dextrine), modified corn starch, maize starch, dried starch sodium starch glycolate, mannitol, sorbitol, silicon dioxide, microcrystalline cellulose, croscarmellose sodium, polyvinylpyrrolidone, polyvinylalcohol, hydroxypropylcellulose, hydroxymethylcellulose, hydroxypropylmethylcellulose, alkylcellulose such as methylcellulose or ethylcellulose, gelatin, cross-linked polyvinylpyrrolidone (PVP) e.g products known under the trademark Avicel®, Filtrak®, Heweten® or Pharmacel®, sodium carboxymethyl starch, magnesium stearate, sodium stearyl fumarate, polyethylene glycol, stearic acid, hydrogenated vegetable oil, glyceyl behenate and talc, colloidal silica e.g known under the trademark Aerosil®, tribasic calcium phosphate, and mixtures thereof.

Further excipients are disclosed in “Handbook of Pharmaceutical excipients”, 2^{nd} Ed., 1994, American Pharmaceutical Association, Washington, ISBN 0 91730 66 8, by Wade A., Weller PJ.).

Another goal is to provide methods of preparing the improved composition according to the invention. The pharmaceutical compositions may be manufactured by various techniques available to the skilled man.

One preferred process comprises the steps of preparing granules of the active ingredient together with the methacrylic polymer and then compressing said granules with extra-granular excipients, into a tablet.

According to a preferred process of the invention, a powder of the active ingredient with the methacrylic polymer is prepared in a first step by conventional processes such as dry spraying, before it is used for the preparation of granules. This preliminary step has the advantage of providing a very fine powder of the active ingredient with the methacrylic polymer which is easier to process in the granulating step.

Granules manufacture can take place according to various techniques, such as fluidized bed technology, tank mixing, or crushing compacts previously manufactured. Granules are optionally, but not necessarily, compressed into tablets.

One embodiment includes the steps of dry mixing the components, compressing into a compressed form and crushing said compressed form into granules. An alternative embodiment includes the step of granulating the components in a fluid bed with a binder.

When the granule obtained (whether subsequently coated or not) is compressed to form tablets, this step can be implemented using any conventional technique which is suitable, for example using alternating or rotating compressing machines.

Direct compression is also available for the manufacture of the instant pharmaceutical compositions.

Coating techniques are also available for the manufacture of a final coated pharmaceutical composition.

The compositions of the invention may further contain a stabilizing amount of a basifying agent such as disclosed in U.S. Pat. No. 5,180,589, U.S. Pat. No. 5,686,104 or a buffering agent such as disclosed in US2007026026. Examples of such stabilizers are inorganic metal oxides and hydroxides, especially magnesium oxide, aluminum oxide, alkali metal hydroxides such as sodium hydroxide, potassium hydroxide or lithium hydroxide and alkaline earth metal hydroxides such as calcium hydroxide or magnesium hydroxide, and any basic inorganic pharmaceutically acceptable calcium, magnesium, aluminum or lithium salt, or mixtures thereof. Examples of these salts are calcium carbonate, calcium hydroxide, magnesium carbonate, magnesium hydroxide, magnesium silicate, magnesium aluminate, aluminum magnesium hydroxide, lithium hydroxide or calcium hydroxide, or mixtures thereof; and buffers such as sodium or potassium citrate, sodium phosphate, dibasic sodium phosphate, calcium carbonate, hydrogen phosphate, phosphate, sulphate, sodium or magnesium carbonate, sodium ascorbinate, benzoate, sodium or potassium hydrogen carbonate, lauryl sulphate, or mixtures thereof. Preferably, the buffering agent includes a phosphate, more preferably a potassium phosphate or sodium phosphate buffering agent. Such a buffering agent, if present, will typically form about 10 weight percent to about 80 weight percent, preferably about 30 weight percent to about 70 weight percent, and more preferably about 40 weight percent to about 60 weight percent, of the total weight of the composition

The pharmaceutical compositions of the present invention may also contain any pharmaceutically acceptable organic acids such as malic acid or tartaric acid. Amounts by weight of release enhancer can be selected in the range of 0.05 to 30%, preferably, 10 to 20% and more preferably between 10 and 15%.

The invention also provides methods for the treatment of hypertension, diabetic nephropathy and congestive heart failure, comprising the step of orally administering to a patient in need of such treatment a therapeutically effective unit dosage form of the invention.

Dissolution profiles are given for the dissolution of compositions comprising an equivalent of 160 mg valsartan in 300 ml buffer solution.

### EXAMPLES

The following examples illustrate the invention without limiting it.

### Example 1

The following formulations are prepared:

| Ingredient (mg) | Batch 5920 | Batch 5914 | Batch 5460 |
|---|---|---|---|
| Valsartan | 160 | 160 | 160 |
| Eudragit E 100 | 220 | 267 | 320 |
| Tartaric acid NF | 50 | 75 | 75 |
| Kollidon 90 F | — | — | 40 |
| Aerosil ® 200 | — | — | 5 |
| Sodium stearyl fumarate | 7 | 10 | 8 |

| Ingredient (mg) | Batch 5257 |
|---|---|
| Valsartan | 320 |
| Avicel PH101 | 300 |
| Magnesium Oxide | 100 |
| Methocel E3lv | 50 |
| Tween 80 | 8 |
| Polyplasdone XL | 100 |
| Sipernat 22 | 50 |
| Sodium stearyl fumarate | 8 |

Dissolution tests are carried out, in an apparatus according to the US Pharmacopeia XXIV, type I basket (10 Mesh), at 75 rpm, in a dissolution medium comprised of acetate buffer at pH 4.0.

The results (expressed in dissolved fraction in %) are the following:

| Time | 5914 | 5460 | 5920 | 5257 | |
|---|---|---|---|---|---|
| (min.) | (267 mg) | (320 mg) | (220 mg) | (0 mg) | Diovan |
| 0 | 0.0 | 0 | 0 | 0.0 | 0 |
| 10 | 5.4 | 5.6 | 0.8 | 5.5 | 11.7 |
| 20 | | 18.1 | | | |
| 30 | 17.2 | 29.8 | 2.4 | 21.4 | 25.5 |
| 60 | 36.4 | 60.1 | 5.4 | 32.8 | 38.3 |
| 90 | | 82.5 | | 37.1 | 45.1 |
| 120 | 70.0 | 95.1 | 37.6 | | 49.0 |
| 150 | 91.0 | 97.1 | | | 51.3 |
| 180 | 97.2 | | | | 52.7 |
| 240 | 97.8 | | 80.6 | | 54.4 |

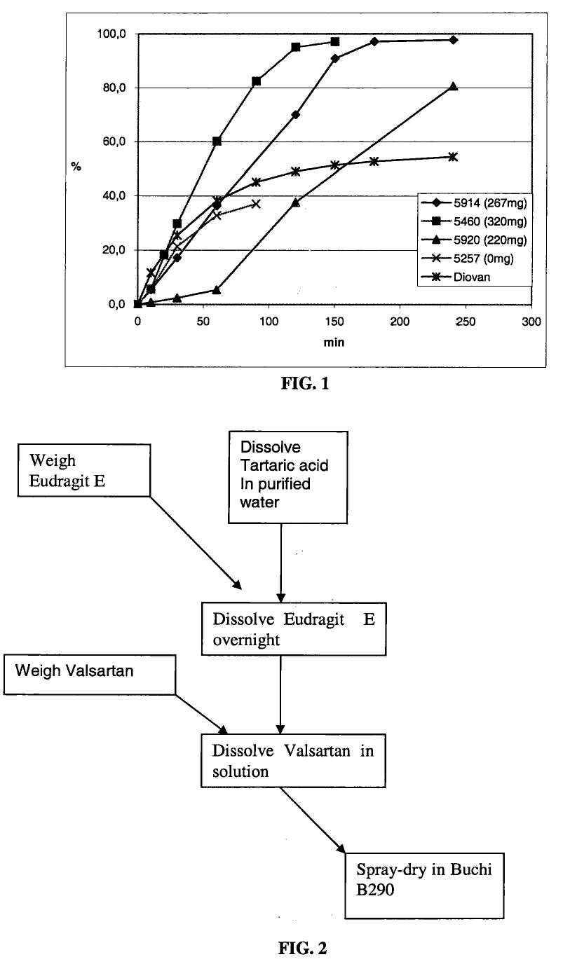

Results clearly show that the dissolution in the case of the invention is far different from the prior art. FIG. 1 is a representation of these results.

### Example 2

The following formulation was prepared:
Spray-dried active.

| Ingredient (mg) | mg/tablet |
|---|---|
| Valsartan | 160 |
| Eudragit E 100 | 320 |
| Tartaric acid NF | 75 |
| Purified water* | 2100 |
| Total | 555 |

*Evaporated during process

Core (granulation/compression)

| | Fluid-bed | |
|---|---|---|
| Ingredient (mg) | granulation | Dry granulation |
| Spray-dried active | 555 | 555 |
| powder | | |
| Kollidon 90 F | 40 | |
| Aerosil 200 | 5 | 5 |
| Purified water* | 810 | |
| Sodium stearyl fumarate | 8 | 8 |
| Total | 608 | 568 |

*Evaporated during process

| | Fluid-bed | | | |
|---|---|---|---|---|
| Core Characteristics | granulation | | Dry granulation | |
| Die size | 19.0 × 10.7 | mm | 16.7 × 8.6 | mm |
| Tablet Thickness | 6.00 | mm | 6.05 | mm |
| Hardness | 16 | Kp | 15 | Kp |

Coating

| Ingredient (mg) | mg/tablet |
|---|---|
| Opadry II White | 20 |
| Carbowax 1450 | 2 |
| granular | |
| Purified water* | 198 |
| Total | 22 |

*Evaporated during process

In a first step, an aqueous solution was prepared by dissolving Eudragit E overnight with a solution of tartaric dissolved in purified water. Valsartan was then added to the resulting mixture and the final solution was spray-dried in Buchi B290, leading to a very fine powder.

The resulting powder was granulated in a fluid-bed Glatt GPCG1 (limits of detection <2%), using a povidone solution (Kollidon K90 in water).

Alternatively, the spray-dried active was dry-granulated. The powder was blended with sodium stearyl fumarate, before it was compacted in Vector TFC-labo, and the resulting compacted powder was passed in an oscillating granulator (20 Mesh).

Sodium stearyl fumarate was added to the resulting granules of any one of the two granulating processes before the mixture was blended with lubricants for 10 minutes in Turbula and compressed with 19.0×10.7 mm tooling on Manesty Betapress.

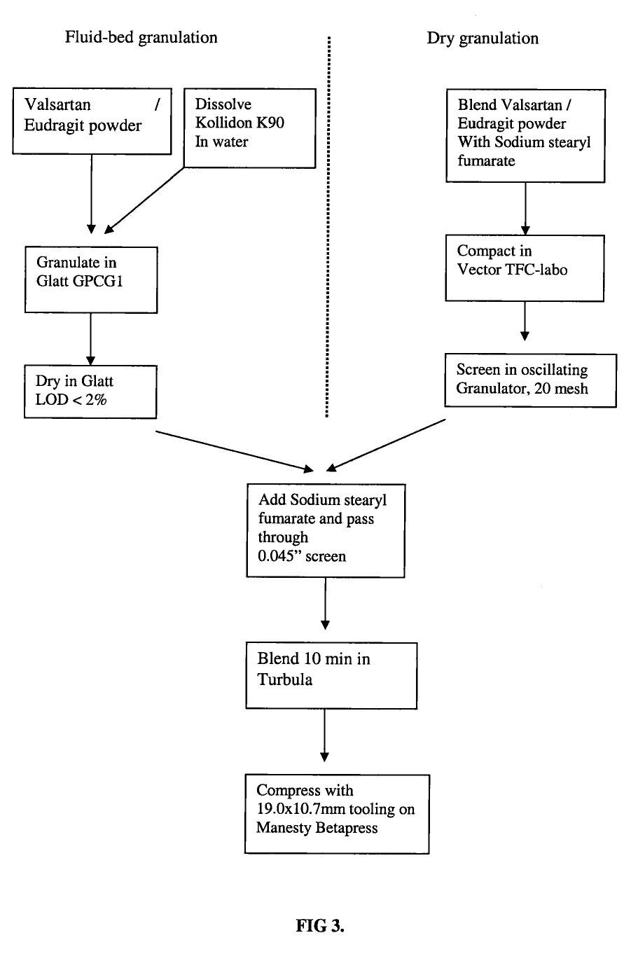

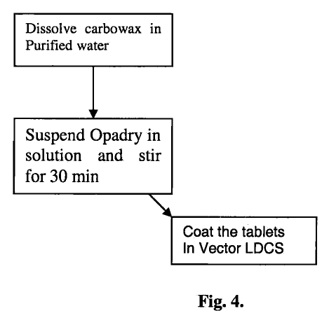

Finally, carbowax is dissolved in purified water and added to opadry. The suspension was stirred for 30 minutes, and the preceding tablets were coated in vector LDCS. The process is summarized in FIGS. 2 to 4.

1. A sartan or salt thereof composition having a dissolution in acetate buffer at pH 4.0 using USP type I method basket 10 Mesh at 75 rpm, of at least 20% after 90 minutes, 50% after 160 minutes, and 70% after 240 minutes.

2. The composition of claim 1 having a dissolution in acetate buffer at pH 4.0 using USP type I method basket 10 Mesh at 75 rpm, of at least 40% after 90 minutes, 70% after 160 minutes, and 80% after 240 minutes.

3. The composition of claim 1 having a dissolution in acetate buffer at pH 4.0 using USP type I method basket 10 Mesh at 75 rpm, of at least 60% after 90 minutes, 80% after 160 minutes and 90% after 240 minutes.

4. The composition of anyone of claim 1, comprising between 10 and 40% wt of sartan.

5. The composition of anyone of claim 1, wherein the sartan is selected from the group consisting of valsartan, candesartan, eprosartan, telmisartan, losartan, irbesartan, and olmesartan, and salts thereof.

6. The composition of claim 1, in a tablet form.

7. The composition according to claim 1, wherein the sartan or salt thereof is valsartan.

8. The composition of claim 7, having a dissolution in acetate buffer at pH 4.0 using USP method basket 10 Mesh at 75 rpm, of at least 40% after 90 minutes, 70% after 160 minutes, and 80% after 240 minutes.

9. The composition of claim 7, having a dissolution in acetate buffer at pH 4.0 using USP method basket 10 Mesh at 75 rpm, of at least 60% after 90 minutes, 80% after 160 minutes, and 90% after 240 minutes.

10. The composition of anyone of claim 7, in a tablet form.

11. A sartan or salt thereof composition comprising at least one aminoalkyl methacrylate copolymer dispersed therein.

12. The composition of claim 11, wherein the aminoalkyl methacrylate copolymer is dimethyl aminoethyl methacrylate.

13. The composition of claim 11, comprising between 10 and 80% wt of aminoalkyl methacrylate copolymer.

14. The composition of claim 11, comprising between 40 and 60% wt of aminoalkyl methacrylate copolymer.

15. The composition of claim 11, comprising between 10 and 40% wt of sartan.

16. The composition of claim 11, wherein the sartan is selected from the group consisting of valsartan, candesartan, eprosartan, telmisartan, losartan, irbesartan, and olmesartan, and salts thereof.

17. The composition of claim 11, under a tablet form.

18. The composition of claim 11, wherein the sartan or salt thereof is valsartan.

19. The composition of claim 18, wherein the aminoalkyl methacrylate copolymer is dimethyl aminoethyl methacrylate.

20. The composition of claim 18, comprising between 10 and 40% wt of valsartan.

21. The composition of claim 18 comprising between 10 and 80% wt of aminoalkyl methacrylate copolymer.

22. The composition of claim 18, under a tablet form.

23. A method for the treatment of hypertension, diabetic nephropathy or congestive heart failure, comprising the step of orally administering to a patient in need of such treatment a therapeutically effective unit dosage of sartan or salt thereof composition having a dissolution in acetate buffer at pH 4.0 using USP type I method basket 10 Mesh at 75 rpm, of at least 50% after 160 minutes.

24. A method for the treatment of hypertension, diabetic nephropathy or congestive heart failure, comprising the step of orally administering to a patient in need of such treatment a therapeutically effective unit dosage of a sartan or salt thereof composition comprising at least one aminoalkyl methacrylate copolymer dispersed therein.

25. A method for the manufacture of a sartan composition, comprising the steps of (i) preparing granules comprising a sartan and at least one aminoalkyl methacrylate copolymer mixed therewith, and (ii) compressing the resulting granules into a tablet.
